(19) United States
(12) Patent Application Publication
(10) Pub. No.: US 2023/0158220 A1
(43) Pub. Date: May 25, 2023

(54) PERITONEAL DIALYSIS PRESSURE SENSING SYSTEMS AND METHODS FOR AIR DETECTION AND ULTRAFILTRATION MANAGEMENT

(71) Applicants: BAXTER INTERNATIONAL INC., Deerfield, IL (US); BAXTER HEALTHCARE SA, Glattpark (Opfikon) (CH)

(72) Inventors: Oskar Erik Frode Styrbjorn Fallman, Lund (SE); Michael Pettersson, Malmo (SE); Jimmie Marcus Axel Hansson, Limhamn (SE)

(21) Appl. No.: 17/984,838

(22) Filed: Nov. 10, 2022

Related U.S. Application Data
(60) Provisional application No. 63/356,332, filed on Jun. 28, 2022.
(60) Provisional application No. 63/283,019, filed on Nov. 24, 2021.

(51) Int. Cl. A61M 1/28; A61M 5/172

(52) U.S. Cl. CPC A61M 1/282; A61M 5/1723; A61M 2205/3331

(57) ABSTRACT

A fluid delivery system includes a fluid pump; a pressure sensor for sensing pressure of fluid pumped by the fluid pump, wherein an output from the pressure sensor varies depending upon whether medical fluid or air is pumped during a pump stroke of the medical fluid pump; and a control unit configured to use the output from the pressure sensor to determine whether medical fluid or air is present during the pump stroke.

### PRIORITY CLAIM AND CROSS-REFERENCE TO RELATED APPLICATIONS

This application claims priority to and benefit of U.S. Provisional Patent App. No. 63/356,332 filed Jun. 28, 2022, titled PERITONEAL DIALYSIS PRESSURE SENSING SYSTEMS AND METHODS FOR AIR DETECTION AND ULTRAFILTRATION MANAGEMENT and U.S. Provisional Patent App. No. 63/283,019 filed Nov. 24, 2021, titled PERITONEAL DIALYSIS PRESSURE SENSING SYSTEMS AND METHODS FOR INLINE HEATER OVERHEATING PREVENTION AND LEVEL SENSING, the entire contents of which are incorporated by reference herein and relied upon.

### BACKGROUND

The present disclosure relates generally to medical fluid treatments and in particular to the heating of treatment fluid during dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid or PD fluid, into a patient's peritoneal chamber via a catheter. The PD fluid comes into contact with the peritoneal membrane in the patient's peritoneal chamber. Waste, toxins and excess water pass from the patient's bloodstream, through the capillaries in the peritoneal membrane, and into the PD fluid due to diffusion and osmosis, i.e., an osmotic gradient occurs across the membrane. An osmotic agent in the PD fluid provides the osmotic gradient. Used PD fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used PD fluid to drain from the patient's peritoneal cavity. The patient then switches fluid communication so that the patient catheter communicates with a bag of fresh PD fluid to infuse the fresh PD fluid through the catheter and into the patient. The patient disconnects the catheter from the fresh PD fluid bag and allows the PD fluid to dwell within the patient's peritoneal cavity, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh PD fluid and to a fluid drain. APD machines pump fresh PD fluid from a dialysis fluid source, through the catheter and into the patient's peritoneal chamber. APD machines also allow for the PD fluid to dwell within the chamber and for the transfer of waste, toxins and excess water to take place. The source may include multiple liters of dialysis fluid, including several solution bags.

APD machines pump used PD fluid from the patient's peritoneal cavity, though the catheter, to drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

Dialysis fluid or treatment for HD, HF, HDF and PD is typically heated prior to being delivered to a dialyzer (HD, HDF), blood line (HF, HDF) or the patient (PD). The dialysis fluid is typically heated to body temperature or 37° C. so that the patient does not experience a thermal shock when the dialysis fluid comingles with the patient's blood or is delivered to the patient's peritoneal cavity. One type of dialysis fluid heater is an inline dialysis fluid heater, which heats the dialysis fluid as it passes through the inline heater. Inline heaters are advantageous because they operate online as treatment is taking place and do not require a separate amount of time offline from the treatment. One drawback to online heating however is that if there is no dialysis fluid flowing when the inline heater is powered, the inline heater may overheat.

There is accordingly a need for an effective, low cost way of preventing or mitigating overheating in an inline heater due to a no or low flow condition. It is also desirable to reduce the amount of hardware in the machine and instead use existing hardware for multiple purposes. For example, a need exists to use existing hardware instead of additional sensors, such as level sensors and pump actuation sensors.

### SUMMARY

The present disclosure involves the use of an inline heater in a dialysis machine, which may be any type of dialysis machine, such as a peritoneal dialysis (“PD”) machine, hemodialysis (“HD”) machine, hemofiltration (“HF”) machine, hemodiafiltration (“HDF”) machine or continuous renal replacement therapy (“CRRT”) machine. The inline heater heats dialysis fluid as it flows through the heater towards the patient (PD), dialyzer (HD, HDF), or blood line (HF, HDF, CRRT) for treatment. The inline heating method is opposed to a batch heater commonly used with PD for heating a bag of dialysis fluid prior to being delivered for treatment. The inline heater of the present disclosure is advantageous because it does not require the footprint involved with maintaining a bag for batch heating. The inline heater also heats the dialysis fluid as it is needed, eliminating the need for a heating period prior to beginning treatment. The present disclosure is also applicable to other devices that may use inline heating, such as water purification units, dialysis fluid preparation units and blood warmers.

The inline heater of the present disclosure is disadvantageous from one standpoint in that if it is attempted to heat dialysis fluid while no dialysis fluid is flowing, the inline heater can overheat. A flow switch may be placed ahead of the inline heater to make sure that flow is present as a condition for energizing the heater. Flow switches add cost however and can become stuck or otherwise not function properly.

The inline fluid heating system of the present disclosure in one primary embodiment involves the use of an already present pressure sensor to detect movement or actuation of the dialysis fluid pump, which presumably means dialysis fluid is flowing through the inline heater. The dialysis fluid pump of the dialysis (or other) machine is of a type that causes a pressure ripple over every stroke, which the pressure sensor detects. The signal from the pressure sensor is cyclical and includes upper and lower peaks that transition over a regular frequency when the dialysis fluid pump pumps at a constant flowrate. The amplitude and frequency of the pressure wave varies for different flowrates. The compliance of the dialysis system also affects the shape of the pressure wave. For example, more air in the dialysis fluid may lower peak to peak pressure reading values.

The system of the present disclosure in one embodiment configures or programs a control unit, e.g., the control unit of a PD machine or other type of unit, to use the sensed pressure oscillations to assume that there is PD or other fluid flow through the inline heater to thereby provide an enable signal for powering the inline heater. The heater enable signal may be created by bandpass filtering the pressure signal and then using a peak detector and a level detector. The heater enable signal for powering the heater may be a square wave or on/off type signal.

In a second primary embodiment, it is contemplated to use a combination of signals to determine (i) whether the pump is actually being actuated and if so (ii) whether the pump is actually moving fluid. If both are true, then the control unit sends an enable signal allowing the inline heater to be powered. The control unit of the PD machine or other type of unit in an embodiment includes a control side that actually controls the components of the PD machine or other type of unit and a protective side that makes sure the components are operating properly. A pump tachometer is provided for outputting to the protective side in one embodiment to count each turn of the PD or other fluid pump and verify that the pump is actually turning. An existing pressure sensor, which may be part of the control side of the control unit, is used as discussed above to ensure that PD or other fluid is actually being pumped. The output of the existing pressure sensor is used as a verification signal to verify that there is PD (or other) fluid flow and that the pump is not pumping air. When the pump is pumping fluid, a distinct pressure ripple is sensed by the pressure sensor. If air is present, the pressure ripple is not sensed.

The control unit in an embodiment bandpass filters the pressure signal and adds a threshold detector to the signal resulting in a pulse signal, which may be a transistor-transistor logic (“TTL”) level pulse signal or other suitable signal. The microprocessor of the control unit determines if the pulse signal is detected while the PD or other fluid pump is running, which is known from the tachometer output. The processor may for example determine if there are pulses coming from the pressure sensor circuit and determine if the pulses comply with a commanded pump stroke speed before turning on or initiating the heater enable signal. If the pulse signal is sensed and matches the commanded pump stroke speed, then the control unit sends the heater enable signal. If the pulse signal is not detected, or a pulse signal not meeting a commanded pump stroke speed is detected, meaning that air may be present in the system, then the heater enable signal is not provided.

In an alternative embodiment, if the pulse signal is detected, the control unit takes no action and a relay on a heater board of the control unit remains in a state that allows power to the heater. If the pulse signal is not detected, the control unit opens the relay on the heater board, which cuts power to the heater.

In a third primary embodiment, the output of the pressure sensor is used to detect how much fluid resides in an airtrap. The PD machine or other type of unit may provide an airtrap that serves to hold a bolus of PD or other fluid if needed and to also provide a place where fluid flow is relatively stagnant so that air may be removed from the PD or other fluid via buoyance. Airtraps typically operate with level sensors that output so that high and low levels of PD or other fluid can be set. The airtrap can be filled until the PD or other fluid reaches the upper level sensor. The airtrap can be drained until the PD or other fluid reaches the lower level sensor.

It has been found that the amplitude of the pressure ripple sensed by the pressure sensor varies depending on how full the airtrap is with PD or other fluid. The greater the airtrap is filled, the greater the amplitude of the pressure ripple. A relationship between pressure signal amplitude and airtrap fluid level is in one embodiment determined via a polytropic process and is stored in the control unit of the PD machine or other type of unit. Here, the compliance of the airtrap is expressed by the equation pVⁿ=C. Here, p is the pressure of the gas or air in the airtrap, which may be measured by a pressure sensor of the fluid delivery system. V is the volume of the air or gas in the airtrap, while C is a constant correlated to the chamber compliance. The exponent n is the polytropic index, which in the present system may be assumed to be isentropic, which is good assuming that the pumping of the PD or other fluid itself does not heat the air or gas in the airtrap significantly. For an isentropic process, n=Cₚ/C_{y}, wherein Cₚ and Cᵥ are the heat capacity for air or other gas at constant pressure and constant volume, respectively. For air, n=1.4 for the typical temperature range associated with the present system. Thus, the volume of the chamber may be calculated at a given time using the relationship V=(C/p)^{1/1.4}. Here, C is correlated to the chamber compliance, which affects the pressure amplitude (p) via a correction factor due to the overall compliance affecting the fluid delivery system. The volume V of air or gas in the airtrap varies as the measured pressure amplitude changes.

A relationship between pressure signal amplitude and airtrap fluid level in an alternative embodiment is determined empirically and is stored in the control unit of the PD machine or other type of unit. The relationship may be specific to each PD machine or other type of unit, e.g., determined at the factory. Or, there may be a general relationship that is used for a plurality of PD machines or other units. The control unit of the PD machine or other type of unit uses the relationship to determine how much PD or other fluid resides in the airtrap. The control unit may then manipulate the valves around the airtrap to raise or lower the PD or other fluid level in the airtrap to reach a desired or preset level.

In a fourth primary embodiment of the present disclosure, the control unit of the fluid delivery system uses the output of a pressure sensor (e.g., located between the fluid pump and the patient, and/or any other pressure sensor that can detect the pressure supplied by the fluid pump) to determine if air is present within the fluid pump during a patient drain stroke (or a patient fill stroke). It should be appreciated for the fourth primary embodiment that the pressure sensor may be located in varying places along the fluid lines and that the outputs from multiple pressure sensors may be taken into account when looking for air.

The control unit in one embodiment includes an air detection circuit that is configured to detect air by analyzing peak to peak sinusoidal pressure wave values outputted by the one or more pressure sensor. The presence of air increases compliance in the fluid path being sensed and thus dampens the peak to peak values from the pressure sensor. The control unit may accordingly look for a threshold decrease in peak to peak values to determine that air is present. In one implementation, if the control unit determines, based on the analysis of the peak to peak outputs of the one or more pressure sensor, that a stroke of the fluid pump has moved air instead of medical fluid, e.g., PD fluid, then that stroke is not counted in an overall volume of fluid moved determination, e.g., for a patient fill or patient drain during a PD treatment. Conversely, if the control unit determines, based on the analysis of the outputs of the one or more pressure sensor, that a stroke of the pump has actually moved medical fluid, e.g., PD fluid, then that stroke volume is counted in the overall volume of fluid moved determination.

In light of the disclosure set forth herein, and without limiting the disclosure in any way, in a first aspect of the present disclosure, which may be combined with any other aspect, or portion thereof, a fluid delivery system includes a fluid pump; an inline heater for heating fluid pumped by the fluid pump; a pressure sensor for sensing pressure of fluid pumped by the fluid pump; and a control unit configured to use a signal from the pressure sensor to determine whether to allow the inline fluid heater to be powered.

In a second aspect of the present disclosure, which may be combined with any other aspect, or portion thereof, the control unit is configured to use the signal from the pressure sensor additionally to control a pressure of fluid pumped by the fluid pump.

In a third aspect of the present disclosure, which may be combined with any other aspect, or portion thereof, the fluid delivery system further includes an airtrap in fluid communication with the fluid pump and the pressure sensor, and wherein the control unit is configured to use the signal from the pressure sensor additionally to control a level of fluid within the airtrap.

In a fourth aspect of the present disclosure, which may be combined with any other aspect, or portion thereof, the control unit is configured to filter the signal from the pressure sensor into an enable signal that allows the control unit to cause the inline fluid heater to be powered.

In a fifth aspect of the present disclosure, which may be combined with any other aspect, or portion thereof, the control unit is configured to bandpass filter the signal from the pressure sensor into the enable signal.

In a sixth aspect of the present disclosure, which may be combined with any other aspect, or portion thereof, the control unit is configured to not cause the inline fluid heater to be powered if the enable signal is not present.

In a seventh aspect of the present disclosure, which may be combined with any other aspect, or portion thereof, the control unit includes a heater protection circuit that opens a relay to depower the inline fluid heater if the signal from the pressure sensor indicates inadequate flow through the inline fluid heater.

In an eighth aspect of the present disclosure, which may be combined with any other aspect, or portion thereof, the pressure sensor is a first pressure sensor and which includes a second pressure sensor for sensing pressure of fluid pumped by the fluid pump, and wherein the control unit is configured to use a signal from at least one of the first or second pressure sensors to determine whether to allow the inline fluid heater to be powered.

In a ninth aspect of the present disclosure, which may be combined with any other aspect, or portion thereof, the fluid pump, inline heater, pressure sensor and control unit are provided as part of a peritoneal dialysis machine, hemodialysis machine, hemofiltration machine, hemodiafiltration machine, continuous renal replacement therapy machine, water purification unit, or a dialysis fluid preparation unit.

In a tenth aspect of the present disclosure, which may be combined with any other aspect, or portion thereof, a fluid delivery system includes a fluid pump operable with a movement detection sensor for detecting whether the fluid pump is in motion; an inline heater for heating fluid pumped by the fluid pump; a pressure sensor for sensing pressure of fluid pumped by the fluid pump; and a control unit configured to use (i) a movement signal from the movement detection sensor and (ii) a pressure signal from the pressure sensor to determine whether to allow the inline fluid heater to be powered.

In an eleventh aspect of the present disclosure, which may be combined with any other aspect, or portion thereof, the control unit is configured to use the signal from the pressure sensor additionally to control a pressure of fluid pumped by the fluid pump.

In a twelfth aspect of the present disclosure, which may be combined with any other aspect, or portion thereof, the fluid delivery system further includes an airtrap in fluid communication with the fluid pump and the pressure sensor, and wherein the control unit is configured to use the signal from the pressure sensor additionally to control a level of fluid within the airtrap.

In a thirteenth aspect of the present disclosure, which may be combined with any other aspect, or portion thereof, the control unit is configured to require (i) the movement signal to indicate that the fluid pump is in motion and (ii) the pressure signal to indicate fluid movement to allow the inline fluid heater to be powered.

In a fourteenth aspect of the present disclosure, which may be combined with any other aspect, or portion thereof, the movement sensor is a tachometer or an encoder.

In a fifteenth aspect of the present disclosure, which may be combined with any other aspect, or portion thereof, the control unit is configured to filter the signal from the pressure sensor into a pulse signal that allows the control unit to cause the inline fluid heater to be powered.

In a sixteenth aspect of the present disclosure, which may be combined with any other aspect, or portion thereof, the control unit is configured to not cause the inline fluid heater to be powered if the pulse signal is not present.

In a seventeenth aspect of the present disclosure, which may be combined with any other aspect, or portion thereof, the fluid pump, inline heater, pressure sensor and control unit are provided as part of a peritoneal dialysis machine, hemodialysis machine, hemofiltration machine, hemodiafiltration machine, continuous renal replacement therapy machine, water purification unit, or a dialysis fluid preparation unit.

In an eighteenth aspect of the present disclosure, which may be combined with any other aspect, or portion thereof, a fluid delivery system includes a fluid pump; an airtrap configured to hold fluid pumped by the fluid pump; a pressure sensor for sensing pressure of fluid pumped by the fluid pump; and a control unit configured to use a pressure signal from the pressure sensor to determine a fluid level within the airtrap.

In a nineteenth aspect of the present disclosure, which may be combined with any other aspect, or portion thereof, the control unit is further configured to determine whether to at least one of fill or drain fluid into or from the airtrap using the determined fluid level.

In a twentieth aspect of the present disclosure, which may be combined with any other aspect, or portion thereof, the control unit uses an amplitude of the pressure signal to determine the fluid level within the airtrap.

In a twenty-first aspect of the present disclosure, which may be combined with any other aspect, or portion thereof, the fluid delivery system includes a fluid valve downstream from the fluid pump and an air valve in fluid communication with the airtrap, and wherein the control unit is configured to close the fluid valve and open the air valve if the amplitude of the pressure signal indicates that the airtrap should be filled.

In a twenty-second aspect of the present disclosure, which may be combined with any other aspect, or portion thereof, the control unit is configured to use the pressure signal from the pressure sensor and a relationship based on a polytropic process to determine the fluid level within the airtrap.

In a twenty-third aspect of the present disclosure, which may be combined with any other aspect, or portion thereof, the control unit is further configured to determine whether an adequate amount of a disinfecting fluid resides within the airtrap using the determined fluid level.

In a twenty-fourth aspect of the present disclosure, which may be combined with any other aspect, or portion thereof, the fluid pump is an inherently accurate piston pump or a gear pump operable with a flowmeter.

In a twenty-fifth aspect of the present disclosure, which may be combined with any other aspect, or portion thereof, a medical fluid delivery system includes a fluid pump; a pressure sensor for sensing pressure of fluid pumped by the fluid pump, wherein an output from the pressure sensor varies depending upon whether medical fluid or air is pumped during a pump stroke of the medical fluid pump; and a control unit configured to use the output from the pressure sensor to determine whether medical fluid or air is present during the pump stroke.

In a twenty-sixth aspect of the present disclosure, which may be combined with any other aspect, or portion thereof, the control unit is configured to use the output from the pressure sensor to determine whether medical fluid, air, or a mixture of medical fluid and air is present during the pump stroke.

In a twenty-seventh aspect of the present disclosure, which may be combined with any other aspect, or portion thereof, the control unit includes an air detection circuit configured to use the output from the pressure sensor to determine whether medical fluid or air is present during the pump stroke.

In a twenty-eighth aspect of the present disclosure, which may be combined with any other aspect, or portion thereof, the air detection circuit includes a bandpass filter configured to filter unwanted signals from the pressure sensor output to form a filtered output.

In a twenty-ninth aspect of the present disclosure, which may be combined with any other aspect, or portion thereof, the air detection circuit includes a comparator configured to analyze the filtered output to determine whether medical fluid or air is present during the pump stroke.

In a thirtieth aspect of the present disclosure, which may be combined with any other aspect, or portion thereof, the air detection circuit includes a counter, and wherein an output from the comparator to the counter goes high and a count at the counter is incremented if medical fluid is determined to be present during the pump stroke.

In a thirty-first aspect of the present disclosure, which may be combined with any other aspect, or portion thereof, the output from the comparator to the counter goes low and the count at the counter is not incremented if air is determined to be present during the pump stroke.

In a thirty-second aspect of the present disclosure, which may be combined with any other aspect, or portion thereof, the control unit is configured to multiply the count by a known volume for the stroke to determine at least one volume of fluid pumped over multiple strokes of the fluid pump.

In a thirty-third aspect of the present disclosure, which may be combined with any other aspect, or portion thereof, the at least one volume of fluid pumped is at least one of a patient drain volume or a patient fill volume for a peritoneal dialysis (“PD”) treatment.

In a thirty-fourth aspect of the present disclosure, which may be combined with any other aspect, or portion thereof, the at least one volume of fluid pumped includes both the patient drain volume and the patient fill volume, and wherein the control unit is configured to subtract the patient fill volume from the patient drain volume to determine an amount of ultrafiltration removed from a patient during the PD treatment.

In a thirty-fifth aspect of the present disclosure, which may be combined with any other aspect, or portion thereof, the control unit includes at least one processor, and wherein the at least one processor is configured to increment a count if an output from the comparator indicates that medical fluid is determined to be present during the pump stroke.

In a thirty-sixth aspect of the present disclosure, which may be combined with any other aspect, or portion thereof, the air detection circuit includes a reset input to the counter, the reset input configured to reset the count to zero prior to at least one of a patient drain or a patient fill for a peritoneal dialysis treatment.

In a thirty-seventh aspect of the present disclosure, which may be combined with any other aspect, or portion thereof, the control unit includes at least one processor and at least one memory configured to use the output from the pressure sensor to determine whether medical fluid or air is present during the pump stroke.

In a thirty-eighth aspect of the present disclosure, which may be combined with any other aspect, or portion thereof, the control unit is configured to analyze peak to peak values of the output from the pressure sensor to determine whether medical fluid or air is present during the pump stroke.

In a thirty-ninth aspect of the present disclosure, which may be combined with any other aspect, or portion thereof, the output from the pressure sensor is a raw output, and wherein the peak to peak values are from the raw output.

In a fortieth aspect of the present disclosure, which may be combined with any other aspect, or portion thereof, the output from the pressure sensor is a sinusoidal output, and wherein the peak to peak values are from the sinusoidal output.

In a forty-first aspect of the present disclosure, which may be combined with any other aspect, or portion thereof, the control unit is configured to determine that air is present during the pump stroke if a threshold decrease in peak to peak values of the output from the pressure sensor is detected.

In a forty-second aspect of the present disclosure, which may be combined with any other aspect, or portion thereof, the control unit is configured to determine that medical fluid is present during the pump stroke if a threshold decrease in peak to peak values of the output from the pressure sensor is not detected.

In a forty-third aspect of the present disclosure, which may be combined with any other aspect, or portion thereof, the output from the pressure sensor is for an upstream portion of the pump stroke during a patient drain or a patient fill.

In a forty-fourth aspect of the present disclosure, which may be combined with any other aspect, or portion thereof, the output from the pressure sensor is for a downstream portion of the pump stroke during a patient drain or a patient fill.

In a forty-fifth aspect of the present disclosure, which may be combined with any other aspect, or portion thereof, a medical fluid method includes determining a delta between peak to peak values of an output of a pressure sensor located upstream or downstream of a fluid pump performing a pump stroke; comparing the determined delta to a threshold delta between peak to peak values; and determining that medical fluid is present during the pump stroke if the determined delta is greater than the threshold delta.

In a forty-sixth aspect of the present disclosure, which may be combined with any other aspect, or portion thereof, the medical fluid method includes determining that air or a combination of air and medical fluid is present during the pump stroke if the determined delta is less than the threshold delta.

In a forty-seventh aspect of the present disclosure, which may be combined with any other aspect, or portion thereof, any of the features, functionality and alternatives described in connection with any one or more of FIGS. 1 to 8 may be combined with any of the features, functionality and alternatives described in connection with any other of FIGS. 1 to 8.

In light of the above aspects and disclosure herein, it is accordingly an advantage of the present disclosure to provide a fluid delivery machine, such as a PD machine or other type of machine, having an inline heater that is deenergized upon a no or low flow condition.

It is another advantage of the present disclosure to provide a fluid delivery machine, such as a PD machine or other type of machine, having cost effective inline heater no or low flow protection.

It is a further advantage of the present disclosure to provide a fluid delivery machine, such as a PD machine or other type of machine, having inline heater no or low flow protection that does not require significant additional hardware.

It is yet another advantage of the present disclosure to provide a fluid delivery machine, such as a PD machine or other type of machine, which includes an airtrap, and which may determine a level of dialysis fluid within the airtrap without the use of level sensors.

It is yet a further advantage of the present disclosure to provide a fluid delivery machine, such as a PD machine or other type of machine, which uses one or more pressure sensor for multiple purposes.

Moreover, it is an advantage of the present disclosure to provide a fluid delivery machine, such as a PD machine or other type of machine, which efficiently detects the presence of air being pumped.

Still another advantage of the present disclosure is to provide a fluid delivery machine, such as a PD machine or other type of machine, which improves fluid volume pumped and ultrafiltration accuracy.

Still a further advantage of the present disclosure is to provide a fluid delivery machine, such as a PD machine or other type of machine, which allows for the elimination of a separate sensor used to ensure that a fluid pump is actually actuated when commanded.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic flow diagram illustrating an inline heater in combination with a fluid pump, pressure sensors, temperature sensor, and an airtrap, which may be used in many different types of dialysis modalities and applications, such as peritoneal dialysis (“PD”), hemodialysis (“HD”) machine, hemofiltration (“HF”), hemodiafiltration (“HDF”), continuous renal replacement therapy (“CRRT”), blood warming, water purification and dialysis fluid preparation.

FIG. 2 is a graph illustrating a typical pressure ripple cause by a fluid pump, e.g., dialysis fluid pump.

FIG. 3 is a collection of graphs correlating an inline heater power input enable signal with a filtered pressure ripple signal for a first primary embodiment of the present disclosure.

FIG. 4 is a schematic view of one embodiment of a heater protection circuit usable with system of the present disclosure.

FIG. 5 is a is a collection of graphs including a signal to a control unit and a filtered pressure ripple signal for a second primary embodiment of the present disclosure.

FIG. 6 is a partially sectioned elevation view of one embodiment for a medical fluid, e.g., PD fluid, pump suitable for use in the system and associated methodologies of the present disclosure.

FIG. 7 is a schematic view of one embodiment of a heater protection circuit usable with system of the present disclosure.

FIG. 8 is are various plots over time showing pumping pressures associated with pump strokes in which fluid is pumped versus pump volumes in which air is pumped.

### DETAILED DESCRIPTION

### System Overview

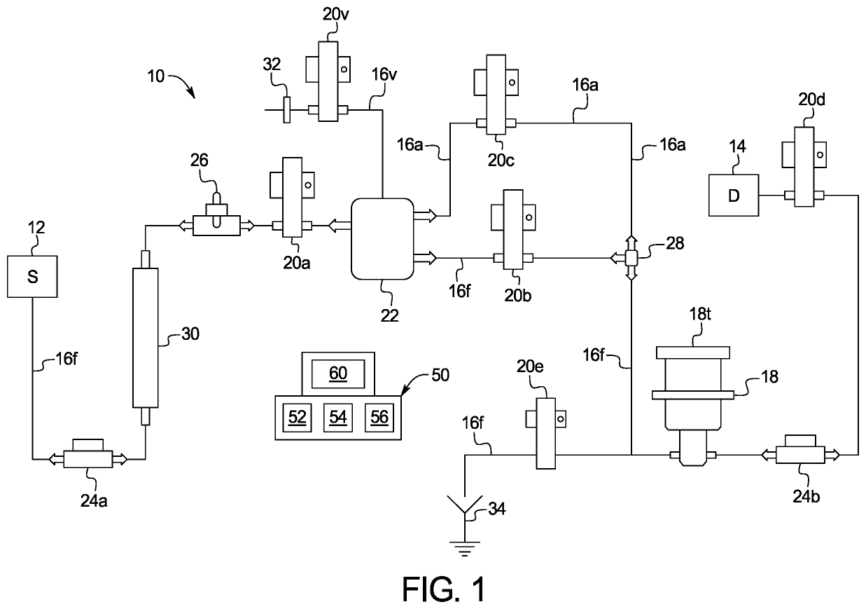

Referring now to the drawings and in particular to FIG. 1, a fluid delivery system 10, such as a peritoneal dialysis (“PD”) having inline heating is illustrated. Fluid delivery system 10 may be used in many medical fluid applications including but not limited to PD, hemodialysis (“HD”) machine, hemofiltration (“HF”), hemodiafiltration (“HDF”), and continuous renal replacement therapy (“CRRT”). Also, while dialysis is a primary focus for fluid delivery system 10, many aspects of the system are not limited to dialysis. For the multiple pressure sensor uses of the present disclosure may be employed in a water purification unit that prepares purified water for the creation of dialysis fluid, and which heats the water either for downstream use or for disinfection. The multiple pressure sensor uses of the present disclosure may be employed alternatively in a dialysis fluid preparation unit that prepares dialysis fluid online, and which heats the dialysis fluid for downstream use or for disinfection.

Fluid delivery system 10 in FIG. 1 includes a fluid source 12, which may be a PD fluid source, HD fluid source, replacement fluid source (HF, HDF, CRRT), or water source for example. Fluid delivery system 10 includes a fluid destination 14, which may be the patient's peritoneal cavity (PD), a dialyzer (HD, HDF), a blood line (HF, HDF, CRRT), a dialysis fluid preparation unit if fluid delivery system 10 is employed in or provided by a water purification unit, or a dialysis machine (e.g., PD cycler) if fluid delivery system 10 is employed in or provided by a dialysis fluid preparation unit. Fluid source 12 and fluid destination 14 may alternatively both be the patient when fluid delivery system 10 is provided as part of a blood warmer.

Fluid from source 12 (which may hereafter be termed dialysis fluid even though the fluid is not limited to same as discussed above) is pumped along a fluid line 16f via a fluid pump 18. Fluid pump 18 may be any type of fluid pump, e.g., a durable (reusable) pump that contacts the dialysis fluid directly, such as an inherently accurate piston or a gear pump operable with a flowmeter. Fluid pump 18 may alternatively have a disposable component, such as a pneumatic pump operating with a disposable cassette, an electromechanical pump operating with a disposable cassette, or a peristaltic pump operating with a peristaltic tube segment.

It is contemplated that there be many different components located along fluid line 16f between fluid source 12 and fluid destination 14, such as one or more valve 20a, 20b, airtrap 22 and various sensors. For ease of illustration fluid delivery system 10 is shown having upstream and downstream pressure sensors 24a and 24b and a temperature sensor 26. The output of one or both pressure sensors 24a and 24b may be used as feedback for controlling fluid pump 18 so as not to exceed a positive or negative pressure limit. The output of the temperature sensor 26 may be used as feedback for controlling the input power to inline heater 30. Temperature sensor 26 is located downstream from inline heater 30 so as to sense the temperature of the dialysis fluid exiting the heater. If desired, an additional temperature sensor (not illustrated) may be located upstream of inline heater 30 to provide feedforward information concerning the temperature of dialysis fluid entering the inline heater.

For purposes of draining a patient in a PD treatment application, system 10 of FIG. 1 includes a drain valve 20e located along a drain line portion of fluid line 16f. As discussed above, fluid destination 14 for PD may be the patient's peritoneal cavity, which is true for a patient fill. During a patient fill, drain valve 20e is closed. During a patient drain, however, fluid destination 14 becomes a fluid source as effluent from the patient's peritoneal cavity is removed via fluid pump 18 to drain, while drain valve 20e under control of control unit 50 is open.

FIG. 1 further illustrates that fluid delivery system 10 of the present disclosure includes a control unit 50, which may be the control unit of a dialysis machine (e.g., PD cycler), water purification unit or a dialysis fluid preparation unit. Control unit 50 in the illustrated embodiment includes one or more processor 52, one or more memory 54 and a video controller 56. Control unit 50 receives, stores and processes signals or outputs from pressure sensors 24a, 24b, temperature sensor 26 and other sensors provided by the machine or unit, such as a conductivity sensor (not illustrated). Control unit 50 uses pressure feedback from pressure sensor 24b to control fluid pump 18 to pump dialysis fluid at a desired pressure or within a safe pressure limit (e.g., within 0.21 bar (three psig) of positive pressure to a patient's peritoneal cavity). Control unit 50 uses temperature feedback from temperature sensor 26 to control inline heater 30 to heat the dialysis fluid to a desired temperature, e.g., body temperature or 37° C. Control unit also causes valves 20a to 20d and 20v (if provided) to open and close according to one or more programmed sequence.

Video controller 56 of control unit 50 interfaces with a user interface 60 of the machine or unit, which may include a display screen operating with a touchscreen and/or one or more electromechanical button, such as a membrane switch. User interface 60 may also include one or more speaker for outputting alarms, alerts and/or voice guidance commands. User interface 60 may be provided with the machine or unit as illustrated in FIG. 1 and/or be a remote user interface operating with control unit 50. Control unit 50 may also include a transceiver (not illustrated) and a wired or wireless connection to a network, e.g., the internet, for sending treatment data to and receiving prescription instructions from a doctor's or clinician's server interfacing with a doctor's or clinician's computer.

Control unit 50 controls the power provided to heater elements, such as two heater elements, of inline heater 30. Control unit 50 may do so by controlling either voltage or current to the heater elements. The more voltage or current supplied, the more power is provided to inline heater 30, and thus more heating of dialysis fluid flowing through inline heater 30 takes place, resulting in a higher dialysis fluid temperature. In one embodiment, control unit 50 controls the voltage from a voltage source (not illustrated) to each heater element. The voltage source may be a 110 to 130 VAC, a 220 to 240 VAC voltage source, or a voltage source supplying direct current voltage to the heater elements.

Inline heater 30 of fluid delivery system 10 is disadvantageous from one standpoint in that if it is attempted to heat dialysis fluid while no dialysis fluid is flowing, inline heater 30 can overheat. A flow switch may be placed ahead of inline heater 30 to make sure that flow is present as a condition for control unit 50 to energize the heater. Flow switches add cost however and can become stuck or otherwise not function properly.

### Pressure Sensing for Heater Control

Fluid delivery system 10 solves the overheating problem in one primary embodiment by configuring control unit 50 to use the output of an already present pressure sensor 24a, 24b to detect movement or actuation of dialysis fluid pump 18, which presumably means dialysis fluid is flowing through inline heater 30. Each of the possible dialysis fluid pumps 18 listed herein causes a pressure ripple over each stroke, which one more pressure sensor(s) 24a, 24b detect(s). With dialysis fluid pump 18 in the position illustrated in FIG. 1 for a patient fill (assuming a PD treatment), the signal ripple from pressure sensor 24a is a negative pressure ripple, while the signal ripple from pressure sensor 24b is a positive pressure ripple. With dialysis fluid pump 18 in the position illustrated in FIG. 1 for a drain (assuming a PD treatment), the signal ripple from pressure sensor 24a is a positive pressure ripple, while the signal ripple from pressure sensor 24b is a negative pressure ripple.

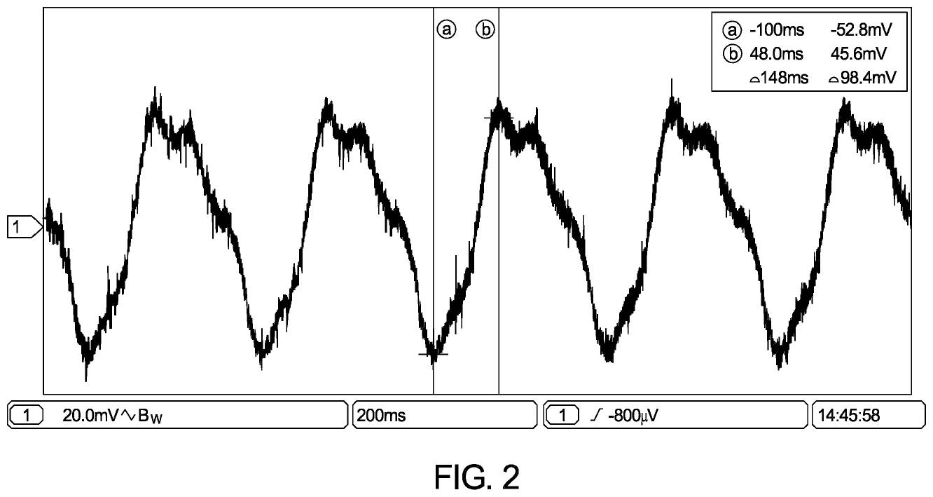

FIG. 2 illustrates that in either the positive or negative pressure ripple scenarios, the signal from the pressure sensor 24a, 24b is cyclical and includes upper and lower peaks that transition over a regular frequency when dialysis fluid pump 18 pumps at a constant flowrate. The amplitude and frequency of the pressure wave varies for different flowrates. The compliance of fluid delivery system 10 (due, e.g., to number of components, length and configuration of the lines, such as fluid line 160 also affects the shape of the pressure wave. For example, more air in the dialysis fluid may lower peak to peak pressure reading values.

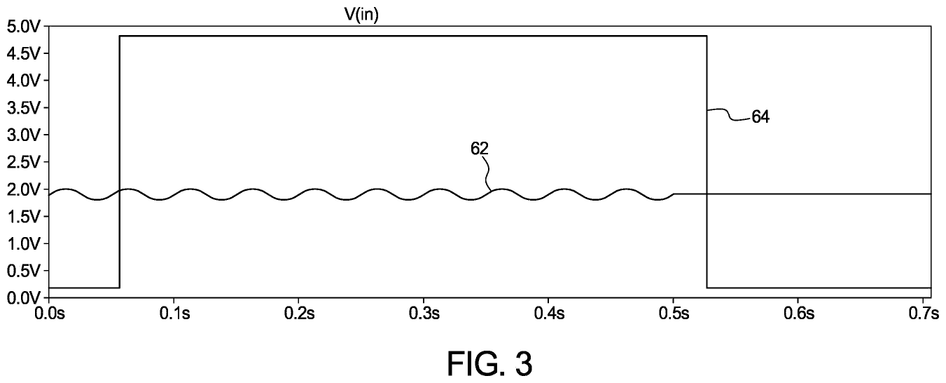

Referring additionally to FIG. 3, fluid delivery system 10 of the present disclosure in one primary embodiment configures or programs control unit 50 of the machine or unit, to use sensed pressure oscillations 62 from pressure sensor 24a and/or 24b to assume that there is fluid flow, e.g., dialysis fluid flow, through inline heater 30 to thereby provide an enable signal 64 for powering the inline heater. Heater enable signal 64 may be created by bandpass filtering pressure signal 62 and then using a peak detector and a level detector. In FIG. 3, pressure signal 62 has not yet been bandpass filtered. Heater enable signal 64 for powering inline heater 30 in the illustrated embodiment is a square wave or on/off type signal.

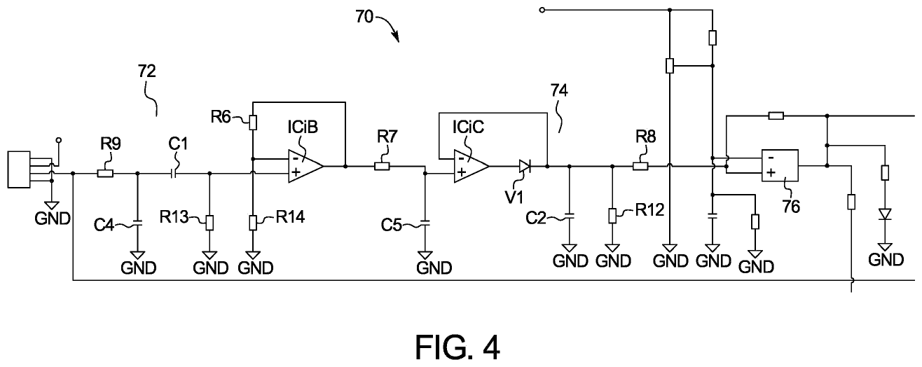

Referring now to FIG. 4, a portion of an overheating heater protection circuit 70 including a bandpass filter 72 and a peak detector 74 is illustrated. It should be appreciated that control unit 50 as discussed herein also includes heater protection circuit 70 and any other heater protection circuitry that may be employed. Control unit 50 includes all supervisory control side and protective side hardware and software and all lower level hardware (including heater protection circuit 70) and software.

It is important to do the bandpass filtering at bandpass filter 72 prior the peak detection via peak detector 74 of heater protection circuit 70. In the illustrated embodiment of FIG. 4, bandpass filter 72 includes a combination of highpass and lowpass filters. Capacitor C1 and resistor R13 form a highpass filter. Capacitor C5 and resistor R7 form a lowpass filter. Together, those filters form a bandpass filter. R6 and R14 are also part of the bandpass filter and set the gain for amplifier ICiB. Capacitor C4 and resistor R9 operate primarily as a noise filter. One reason for bandpass filtering before peak detection is that a direct current (“DC”) signal, e.g., a high pressure DC signal from a closed valve or other flow blockage while pump 18 is running, creating a high pressure and causing an abnormal pressure signal due to no or low fluid flow, is filtered out by the high pass filter portion of bandpass filter 72. To mitigate against offset workpoints (different pressure heights) and to take into consideration the possibility of running pump 18 without actually moving fluid, heater protection circuit 70 removes the DC signal so that only the peak to peak difference in a desired (filtered) range (e.g., 0.5 to 12 Hz) is analyzed to determine if fluid is flowing. Otherwise, peak detector 74 would see the high pressure DC signal as flow as well. Peak detector portion 74 in FIG. 4 includes V1, C2, R12. With the high pressure DC signal filtered out via bandpass filter 72, peak detector 74 only sees pressure spikes in a frequency range of those outputted by pump 18, e.g., around 0.5 to 12 Hz. Heater protection circuit 70 as illustrated in FIG. 4 also includes a level detector 76, which may be a comparator provided after peak detector 74 in circuit 70. The resisters surrounding level detector 76 provide a reference level with respect to ground. The level is compared with an incoming signal from the rest of the circuit to output 1 or 0 depending if the signal is larger or smaller than a set reference level.

Heater enable signal 64 is in one embodiment required for control unit 50 to power inline heater 30. If heater enable signal 64 is not provided or detected, control unit 50 is prevented from powering inline heater 30. If heater enable signal 64 is provided or detected, control unit 50 is allowed to power inline heater 30. Powering inline heater 30 is performed via a heater control algorithm run by control unit 50, which may be a proportional, integral, derivative (“PID”) algorithm that uses feedback from temperature sensor 26 and perhaps an additional upstream temperature sensor (not illustrated).

### Pressure and Tachometer Sensing for Heater Control

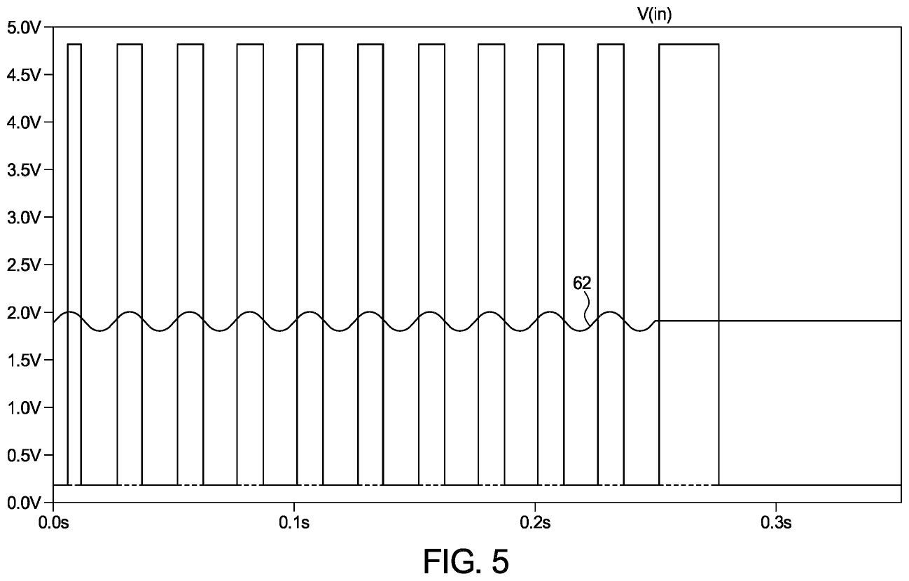

Referring now to FIG. 5, in a second primary embodiment, control unit 50 uses a combination of signals to determine (i) whether fluid pump 18, e.g., dialysis fluid pump, is actually being actuated and if so (ii) whether the pump is actually moving fluid. If both pump actuation and fluid moving are true, control unit 50 sends enable signal 64 allowing inline heater 30 to be powered as described above for the first primary embodiment. Control unit 50 of the PD machine or type of unit of fluid delivery system 10 in an embodiment includes a control side that actually controls the components of the machine or unit and a protective side that makes sure the components are operating properly.

A pump tachometer 18t is provided in one embodiment to count each turn of the fluid pump 18 and verify for the protective side of control unit 50 that fluid pump 18 is actually turning or otherwise actuating. Existing pressure sensors 24a, 24b, which may be part of the control side of control unit 50, is/are used as discussed above to ensure that PD or other fluid is actually being pumped. Control unit 50 uses the output of one or more pressure sensor 24a and/or 24b as a verification signal to verify that there is PD or other fluid flow and that fluid pump 18 is not pumping air. When fluid pump 18 is pumping fluid, e.g., PD or other fluid, a distinct pressure ripple 62 is again sensed by pressure sensor 24a, 24b. If air is present instead, the pressure ripple is not sensed.

Control unit 50 in the second primary embodiment may bandpass filter the pressure signal, like with the first primary embodiment, and add a threshold detector to the signal, resulting in a pulse signal (which may be a transistor-transistor logic (“TTL”) level signal) that is sent to microprocessor 52 of control unit 50. Processor 52 determines if the pulse signal is detected while the PD or other fluid pump is running, which is known to control unit 50 from the output of tachometer 18t. If control unit 50 senses the pulse signal, then the control unit sends the heater enable signal as discussed in connection with the first primary embodiment. If control unit 50 does not detect the pulse signal, meaning air is present in in the fluid components, e.g., fluid line 16f, fluid pump 18, etc., of fluid delivery system 10, then the control unit does not provide the heater enable signal.

### Pressure Sensing for Level Sensing

In a third primary embodiment of the present disclosure, control unit 50 of fluid delivery system 10 uses the output of pressure sensor 24a (and/or any other pressure sensor that can detect the pressure within airtrap 22) to detect how much fluid resides in airtrap 22. It should be appreciated for the third primary embodiment that pressure sensor 24a may be located along any portion of fluid line 16f upstream from fluid pump 18, e.g., upstream from inline heater 30 as illustrated, on either side of temperature sensor 26, on either side of valves 20a, 20b, or on either side of airtrap 22. The PD machine or other type of unit (e.g., water purification unit, dialysis fluid preparation unit) may provide an airtrap 22 to hold a bolus of fluid, e.g., PD fluid, if needed and to also provide a place where fluid flow is relatively stagnant, so that air may be removed from the fluid within airtrap 22 via buoyance.

An air line 16a extends from airtrap 22 to an air valve 20c and from air valve 20c to fluid line 16f via a junction 28. In the illustrated embodiment, a vent line 16v optionally extends from the top of airtrap 22 to a vent valve 20v, which communicates with ambient air via a hydrophobic membrane or filter 32 that filters any air entering air line 16a via vent valve 20v. Vent valve could alternatively be located along air line 16a between vent valve 20v and air valve 20c, however, locating vent valve 20v off the top of airtrap 22 is advantageous from the standpoint that it provides the most protection against hydrophobic membrane or filter 32 coming into contact with PD fluid or other fluid, which could contaminate or affect the integrity of the membrane or filter and possibly block it from allowing air in or out.

Airtraps typically operate with level sensors that output so that high and low levels of PD or other fluid can be set. Airtrap 22 may be filled for example until the PD or other fluid reaches the upper level sensor, e.g., with vent valve 20v open to push air to atmosphere or with air valve 20c open and vent valve 20v closed (or not provided) to push air to a fluid destination 14, such as a drain, via fluid line 16f Airtrap 22 may be emptied for example until the PD or other fluid reaches the lower level sensor, e.g., with vent valve 20v open to pull in ambient air through hydrophobic membrane or filter 32.

It has been found that the amplitude of the pressure ripple sensed by at least one pressure sensor 24a varies depending on how full the airtrap 22 is with PD or other fluid. The greater that airtrap 22 is filled, the greater the amplitude of the pressure ripple sensed by control unit 50. A relationship between pressure signal amplitude and the fluid level of airtrap 22 is in one embodiment determined via a polytropic process and is stored in the control unit 50 of the PD machine or other type of unit. The compliance of airtrap 22 may be expressed by the equation pVⁿ=C. Here, p is the pressure of the gas or air in airtrap 22, which may be measured by pressure sensor 24a of fluid delivery system 10. V is the volume of the air or gas in airtrap 22, while C is a constant correlated to the chamber compliance. The exponent n is the polytropic index, which in the present system may be assumed to be isentropic, which is good assuming that the pumping of the PD or other fluid itself does not heat the air or gas in the airtrap significantly. For an isentropic process, n=Cₚ/C_{y}, wherein Cₚ and Cᵥ are the heat capacity for air or other gas at constant pressure and constant volume, respectively. For air, n=1.4 for the typical temperature range associated with the present system. Thus, the volume of the chamber may be calculated at a given time using the relationship V=(C/p)^{1/1.4}. Here, C is correlated to the chamber compliance, which affects the pressure amplitude (p) via a correction factor due to the overall compliance affecting the fluid delivery system. The volume V of air or gas in the airtrap varies as the measured pressure amplitude changes.

A relationship between pressure signal amplitude and the fluid level within airtrap 22 may be determined alternatively empirically and stored in control unit 50 of the PD machine or other type of unit. The relationship may be specific to each PD machine or other type of unit, e.g., determined at the factory. Or, there may be a general relationship that is used for a plurality of PD machines or other units. Also, in any of the above examples, compliance of the air in airtrap 22 affects the amplitudes of the pressure spikes. Control unit 50 may store a lookup table or a mathematical relationship between the amplitudes detected and the level of fluid, e.g., PD fluid, within airtrap 22.

In any pressure amplitude versus airtrap fluid level relationship embodiment discussed above, control unit 50 of fluid delivery system 10 uses the relationship to determine how much PD or other fluid resides in airtrap 22 based on the output of at least one pressure sensor 24a. Control unit 50 may then manipulate the valves of fluid delivery system 10 to raise or lower the PD or other fluid level in airtrap 22 to reach a desired or preset fluid level. Control unit 50 may raise the fluid level in a plurality of different ways. In a first way, vent line 16v, vent valve 20v and hydrophobic membrane or filter 32 are not used and do not need to be provided. Here, fluid valve 20b is closed, while fluid valves 20a and 20d and air valve 20c are opened to allow fluid pump 18 to pull air from the top of airtrap 22 causing fluid, such as PD fluid, to be pulled from fluid source 12 into airtrap 22, raising the fluid level within airtrap 22. Air is correspondingly pushed down air line 16a into fluid line 16f at junction 28, which can then be pumped to a desired fluid destination 14, e.g., a house drain or drain container. Because the filling of airtrap 22 is here performed in a closed system (no connection to atmosphere), control unit 50 is able to monitor the amplitude of the output pressure ripple from pressure sensor 24a and sense an increase in amplitude until the amplitude rises to where the corresponding fluid level within airtrap 22 is at a desired fluid level. Control unit 50 then causes fluid valve 20b to open and air valve 20c to close, so that the level within airtrap 22 remains constant at the desired level, while fluid pump 18 pumps fluid to continue treatment or other operation.

In a second way for raising the fluid level within airtrap 22, vent line 16v, vent valve 20v and hydrophobic membrane or filter 32 are provided and used. Here, valves 20a and 20c are closed, while fluid valves 20b and 20d and vent valve 20v are opened to allow fluid pump 18 to run in reverse and pull downstream fluid, e.g., PD or other fluid from destination 14 into airtrap 22, raising the fluid level within airtrap 22. Air is correspondingly pushed to atmosphere via vent line 16v, vent valve 20v and hydrophobic membrane or filter 32. Because the filling of airtrap 22 is here performed in an open system (having a connection to atmosphere), control unit 50 is not able to monitor the amplitude of the output pressure ripple from pressure sensor 24a. Instead, control unit 50 relies on (i) the fluid level in airtrap 22 determined prior to opening vent valve 20v using the pressure sensor detection methodology as described herein and (ii) the accuracy of inherently accurate pump 18, e.g., piston pump, or an integrated output from a flowmeter, to know how much fluid, e.g., PD fluid, has been pushed into airtrap 22. Once an amount of fluid accumulated from accurate pump stroke volumes, or an integrated flow meter output, equals an amount needed to raise the fluid level within airtrap 22 to a desired level, control unit 50 causes valve 20a to open and vent valve 20v to close, so that the level within airtrap 22 remains constant at the desired level, while fluid pump 18 pumps now in the normal, forward fluid to continue treatment or other operation.

Control unit 50 may also manipulate the valves of fluid delivery system 10 to lower the PD or other fluid level in airtrap 22 so as to reach a desired or preset fluid level. To lower the fluid level within airtrap 22, vent valve 20v and hydrophobic membrane or filter 32 are again provided and used. Here, upstream fluid valve 20a and air valve 20c are closed, while fluid valves 20b and 20d and vent valve 20v are opened to allow fluid pump 18 to pull fluid, e.g., PD or other fluid, from airtrap 22, lowering the fluid level within airtrap 22. Air is correspondingly pulled in from atmosphere via vent line 16v, vent valve 20v and hydrophobic membrane or filter 32 to backfill the fluid removed from airtrap 22. Because the draining of airtrap 22 is performed in an open system (having a connection to atmosphere), control unit 50 is not able to monitor the amplitude of the output pressure ripple from pressure sensor 24a. Instead, control unit 50 relies again on (i) the fluid level in airtrap 22 determined prior to opening air valve 20c and vent valve 20v using the pressure sensor detection methodology as described herein and (ii) the accuracy of inherently accurate pump 18, e.g., piston pump, or an integrated output from a flowmeter, to know how much fluid, e.g., PD fluid, has been pulled from airtrap 22. Once an amount of fluid accumulated from accurate pump stroke volumes, or an integrated flow meter output, equals an amount needed to lower the fluid level within airtrap 22 to a desired level, control unit 50 causes the upstream fluid valve 20a to open and vent valve 20v to close, so that the level within airtrap 22 remains constant at the desired level, while fluid pump 18 pumps fluid to continue treatment or other operation.

Determining the fluid level within airtrap 22 by monitoring the amplitude of the output pressure ripple from pressure sensor 24a as discussed herein is useful for many reasons in addition to adjusting the fluid level. In one example, with system 10 closed to atmosphere, control unit 50 monitors the amplitude of the output pressure ripple from pressure sensor 24a as discussed herein to verify a volume of a disinfecting fluid within airtrap 22, so that adequate disinfection can be assured.

### Pressure Sensing for Air Detection and Ultrafiltration Management

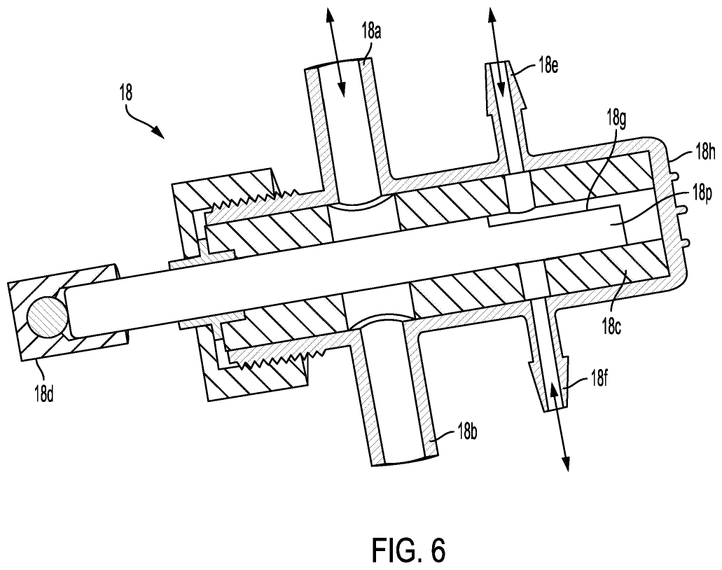

Referring now to FIGS. 6 to 8, in a fourth third primary embodiment of the present disclosure, control unit 50 of fluid delivery system 10 uses the output of pressure sensor 24b (and/or any other pressure sensor that can detect the pressure supplied by pump 18) to determine if air is present within pump 18 during a patient drain stroke (or a patient fill stroke). It should be appreciated for the fourth primary embodiment that pressure sensor 24b may be located along any portion of fluid line 16f between pump 18 and valve 20d. The air detection discussed herein may be performed for one or both of a patient fill and a patient drain.

As discussed herein, pump 18 is in one embodiment a piston pump. FIG. 6 illustrates one example piston pump 18. Piston pump 18 in the illustrated embodiment includes a housing 18h holding a cylinder 18c within which a piston 18p is actuated via a motor (not illustrated), under control of control unit 50, driving a motion coupler 18d coupled to piston 18p, wherein motion coupler 18d converts a rotational motion of the motor to a rotational and translational movement of piston 18p. Housing 18h includes fluid inlet/outlet ports 18e and 18f (bidirectional) and flush flow ports 18a and 18b (bidirectional or stagnant).

Motion coupler 18d moves piston 18p in and out relative to cylinder 18c to create positive and negative pumping pressure, respectively. Motion coupler 18d also rotates piston 18p within cylinder 18c to move fluid from one of ports 18e and 18f, acting as a PD or other fluid inlet port, to the other of ports 18e and 18f, acting as a PD or other fluid outlet port. The distal end of piston 18p includes a cutout or groove 18g forming a flat. The open area formed by groove 18g accepts PD or other fluid at the inlet port 18e or 18f (under negative pressure when piston 18p is retracted within cylinder 18c) and is then rotated to deliver PD fluid at the outlet port 18e or 18f (under positive pressure when piston 18p is extended within cylinder 18c). Groove 18g provides the valve functionality so that dialysis fluid pump 18 can have different flow directions.

The translational and rotational movement of piston 18p within cylinder 18c creates heat and friction. A flush flow of fluid is provided accordingly to lubricate the translational and rotational movement of piston 18p within cylinder 18c. The flush flow of fluid, e.g., reverse osmosis, distilled or deionized water, is provided at flush flow ports 18a and 18b to contact piston 18p as it is moved translationally and rotationally within cylinder 18c. The flush flow of fluid may be circulated or stagnant.

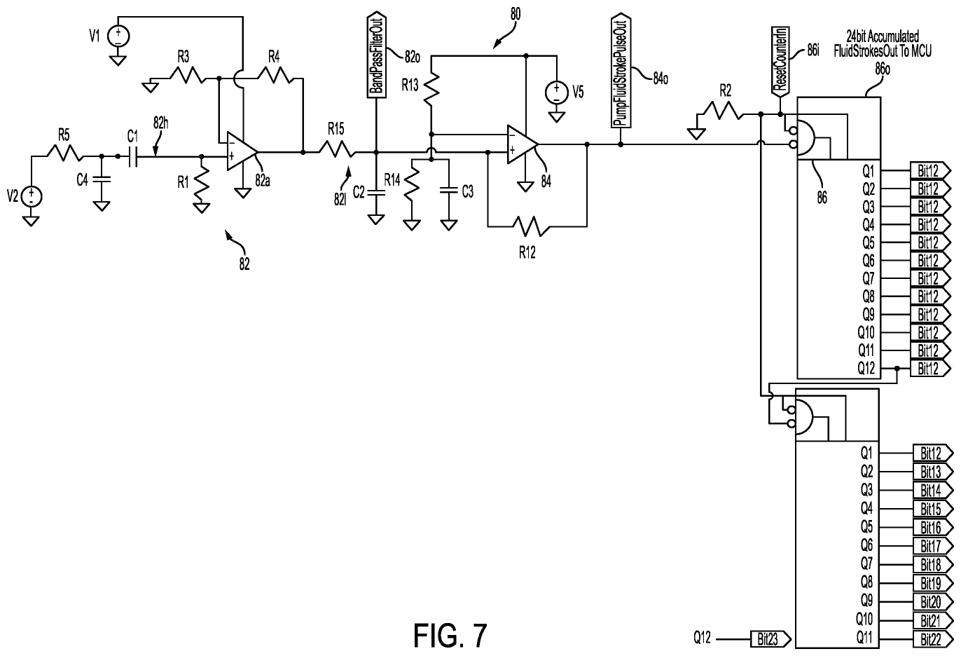

Referring now to FIG. 7, at least a portion of an air detection circuit 80 for detecting air being pumped by pump 18 is illustrated and is provided as part of control unit 50. In the piston pump 18 illustrated in connection with FIG. 6, air detection circuit 80 would detect air versus medical fluid entering the piston pump defined between groove 18g, the end of piston 18p, and the inside wall of cylinder 18c. Here, control unit 50 of system 10 does not simply rely on the fact that pump 18 makes a pump stroke. Control unit 50 of system 10 also looks to the output of pressure sensor 24b to check that the pump stroke has actually moved fluid. If control unit 50 determines, based on the output of pressure sensor 24b, that a stroke of pump 18 has moved air instead of medical fluid, e.g., PD fluid, then that stroke is not counted in an overall volume of fluid moved determination, e.g., for a patient fill or drain during a PD treatment. Conversely, if control unit 50 determines, based on the output of pressure sensor 24b, that a stroke of pump 18 has actually moved medical fluid, e.g., PD fluid, then that stroke volume is counted in the overall volume of fluid moved determination, e.g., for a patient fill or drain during a PD treatment.

Air detection circuit 80 includes a bandpass filter 82. Capacitor C1 and resistor R1 form a highpass filter 82h. Capacitor C2 and resistor R15 form a lowpass filter 82l. Together, those filters form bandpass filter 82. Resistors R3 and R4 are also part of bandpass filter 82 and set the gain for amplifier 82a. Capacitor C4 and resistor R5 operate primarily as a noise filter. With the high pressure DC signal filtered out via bandpass filter 82 at output 82o, a downstream comparator 84 only sees pressure spikes in a frequency range of those outputted by pump 18, e.g., around 0.5 to 12 Hz. Downstream comparator 84 analyzes the filtered pressure signal to determine whether a just completed stroke by pump 18 has pumped fluid, e.g., PD fluid, or air. In one embodiment, the output 84o of comparator 84 is set high, e.g., to 1, if the analysis by comparator 84 of the bandpass filtered signal indicates that the just completed stroke by pump 18 has pumped fluid. The output 84o of comparator 84 is set low, e.g., to 0, if the analysis by comparator 84 of the bandpass filtered signal indicates that the just completed stroke by pump 18 has pumped air.

Air detection circuit 80 in the illustrated embodiment includes a counter 86 to which the high or low, e.g., 1 or 0, output from comparator 84 is sent. Counter 86 is resettable to zero via a reset counter input 86i. For a PD treatment, counter 86 may be reset to zero just prior to the beginning of a patient fill and just prior to the beginning of a patient drain. Counter 86 accumulates counts over each of the patient drain and patient fill. The counts are only incremented when comparator 84 determines that a stroke actually pumped PD fluid. If comparator 84 instead determines that the stroke actually pumped PD air, the low or zero output does not increase the count. In this way, patient drain and patient fill volumes are accumulated more accurately. It should be appreciated that counter 86 of air detection circuit 80 of control unit 50 may be implemented in hardware as illustrated. In an alternative embodiment, the counter (or the function of counting PD fluid strokes) may instead be performed by a supervisory processor 52 of control unit 50.

Control unit 50, e.g., a supervisory processor 52 of the control unit, stores the accumulated counts for each of the patient drain and the patient fill. Control unit 50 also knows the volume pumped per counted stroke, i.e., a stroke that has actually pumped PD fluid. Supervisory processor 52 may therefore accurately determine the total volume of a patient drain (pump stroke volume times number of actual PD fluid movement drain strokes), the total volume of a patient fill (pump stroke volume times number of actual PD fluid movement fill strokes), and the difference between the volumes (total drain volume less total fill volume), which is known as ultrafiltration (“UF”), an important PD parameter for knowing how much accumulated fluid has been removed from the patient over the course of a PD treatment.

In an embodiment, control unit 50 also monitors the counts for each of the patient drain and the patient fill. Here, if control unit 50 sees multiple, sequential low or zero outputs from comparator 84, the control unit determines that there is a sustained leak and causes treatment to stop and user interface 60 to provide an audio, visual or audiovisual alarm letting the patient know that treatment has been paused and to look for the source of a leak, e.g., an incorrectly connected medical fluid, e.g. PD fluid, supply container or source 12.

As shown above, air detection circuit 80 monitors whether or not a stroke of pump 18 has actually moved medical fluid, e.g., PD fluid. In doing so, it effectively provides the information obtained from tachometer 18t. As discussed herein, the output from tachometer 18t is used to confirm that a motor shaft for pump 18 has actually rotated when motor 18 is commanded to do so. If the shaft of motor 18 does not turn when commanded to do so, then an expected or characteristic output from pressure sensor 24b is not detected by air detection circuit 80 and a “no flow” or “motor fault” signal is sent to control unit 50. Air detection circuit 80 accordingly performs the job of tachometer 18t, which may be eliminated for cost purposes. Tachometer 18t may alternatively be used in addition to air detection circuit 80 as an extra safety check.

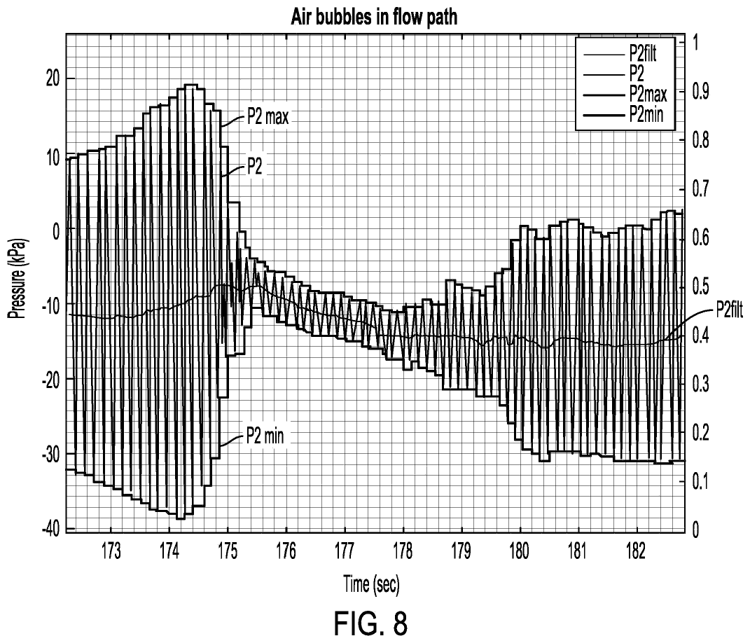

FIG. 8 is a plot showing how air affects the pressure output from pump 18. P2_{filt} is the output from lowpass filter 82l of air detection circuit 80. P₂ is the raw output from pressure sensor 24b in FIG. 1. P₂ₘₐₓ and P₂ₘᵢₙ are plots from peak to peak for the upper peaks and lower peaks, respectively, for P₂, which is the raw output from pressure sensor 24b. Control unit 50 analyzes the peak to peak difference between P₂ₘₐₓ and P₂ₘᵢₙ to determine if full fluid stokes occur. In FIG. 8, control unit 50 (e.g., one or more processor 52 and one or more memory 54) may be used to determine that air (or a mixture of air and PD fluid) is present from about t175 to about t178. A peak P₂ₘₐₓ to peak P₂ₘᵢₙ pressure difference prior to t175 ranges from about 40 kPa to about 55 kPa (5.8 psig to 8 psig). Between t175 to about t178, the peak to peak pressure difference drops so as to range from about 8 kPa to about 20 kPa (1.2 psig to 2.9 psig). Here, control unit 50 may be programmed to look for a change (drop) in peak P₂ₘₐₓ to peak P₂ₘᵢₙ pressure difference of, e.g., at least fifty percent. When the, e.g., fifty percent decrease threshold is met, and for as long as it is met, control unit 50 does not count the associated strokes for accumulating volume (an may cause an alarm or alert to be provided). In an embodiment, a stroke containing partial air and partial PD fluid is not counted. It is contemplated however that over time, as data is accumulated and associated software is optimized, that accurate partial stroke volumes may be ascertained and included in the count, e.g., as a percentage of one stroke multiplied by stroke volume, for accumulating volume.

It should be appreciated however that it is not required that one or more processor 52 and one or more memory 54 of control unit 50 be used to determine that air (or a mixture of air and PD fluid) is present. Instead, air detection circuit 80 of control unit 50 in FIG. 7 may be used to determine that air (or a mixture of air and PD fluid) is present and to count PD fluid volume strokes accordingly. Here, the determination is made purely through hardware. In an embodiment, bandpass filter 82 extracts a peak to peak signal (FIG. 8) without its offset. Peak detection depends on the difference between the high hand low peak values. Counter 86 then only counts strokes with a large enough or threshold delta between the peak values, thus implementing the air detection and accurate PD fluid volume pumped determination in hardware.

Viewing pressure sensor 24b in FIG. 1 and assuming its output to be used to evaluate pump 18 draining patient 14, it may appear as if only negative pressure would be read, not negative and positive pressures as shown in FIG. 8. In one set of circumstances however, where a stroke volume of fluid pump 18, e.g., a dialysis fluid pump, is small (e.g., 0.5 ml) relative to the mass of fluid in the patient line leading to patient 14, the pressure reads negative while the fluid is being pulled during the negative pressure portion of the pump stroke. At the end of the negative pressure portion of the pump stroke, the pulled fluid flow is stopped, causing a positive pressure spike to occur as the fluid backs up against piston 18p of fluid pump 18. Hence, the positive pressures seen in FIG. 8, which is again for a patient drain. The characteristics of the positive pressure spike depend on the compliance of the tubing leading to patient 14 and on the speed of the fluid pump 18. The presence of air significantly increases the compliance within the tube, thus dampening the peak to peak values as illustrated in FIG. 8.

Another set of circumstances in which positive pressures are detected during a patient drain occurs if the patient is positioned above the PD machine. Such patient positioning causes the head height to be positive. Viewing FIG. 1, if the patient residing at fluid destination 14 is located above fluid pump 18, then pressure sensor 24b may see positive pressures even though the fluid pump is creating negative pressure, e.g., a piston pump as in FIG. 6 in which piston 18p is being retracted.

The air determination of system 10 in the embodiment of FIGS. 6 to 8 is not limited to looking at (i) upstream (of fluid pump 18) pressures during a patient drain but may also be used while looking at any one or more of (ii) downstream (of fluid pump 18) pressures during a patient drain (pumping effluent from pump 18 towards drain 34), (iii) downstream pressures during a patient fill (pumping fresh, heated PD fluid from pump 18 towards patient 14), and/or (iv) upstream pressure during a patient fill (pumping fresh, heated PD fluid into pump 18 from fluid source 12). In an embodiment for either patient draining or filling, air detected during the upstream pressure portion of the pump stroke may be confirmed by control unit 50 during the downstream pressure portion of the pump stroke. Here, the peak to peak raw outputs from sensors located both upstream and downstream from fluid pump 18 are analyzed.

It should be understood that various changes and modifications to the presently preferred embodiments described herein will be apparent to those skilled in the art. It is therefore intended that any or all of such changes and modifications may be covered by the appended claims. For example, dialysis fluid pump 18 is illustrated as being downstream from inline heater 30 (for a patient fill), the principles of fluid delivery system 10 apply equally if dialysis fluid pump 18 is located upstream of inline heater 30 (for a patient drain). It should also be appreciated that control unit 50 of fluid delivery system 10 may operate the first and third primary embodiments (inline heating and airtrap) or the second and third primary embodiments (inline heating and airtrap) together and simultaneously using pressure sensors 24a and/or 24b for multiple purposes in addition to their fluid pumping pressure purpose. Also, while tachometer 18t is illustrated and described in connection with the second primary embodiment, another type of movement sensor, such as encoder e.g., incremental or absolute encoder, may be used instead to confirm that fluid pump is being actuated. Further alternatively, the functionality provided by air detection circuit 80 may instead be programmed into one or more processor 52 and one or more memory 54 of control unit 50.

The invention is claimed as follows:

1. A medical fluid delivery system comprising:
a fluid pump;
a pressure sensor for sensing pressure of fluid pumped by the fluid pump, wherein an output from the pressure sensor varies depending upon whether medical fluid or air is pumped during a pump stroke of the medical fluid pump; and
a control unit configured to use the output from the pressure sensor to determine whether medical fluid or air is present during the pump stroke.

2. The medical fluid delivery system of claim 1, wherein the control unit is configured to use the output from the pressure sensor to determine whether medical fluid, air, or a mixture of medical fluid and air is present during the pump stroke.

3. The medical fluid delivery system of claim 1, wherein the control unit includes an air detection circuit configured to use the output from the pressure sensor to determine whether medical fluid or air is present during the pump stroke.

4. The medical fluid delivery system of claim 3, wherein the air detection circuit includes a bandpass filter configured to filter unwanted signals from the pressure sensor output to form a filtered output.

5. The medical fluid delivery system of claim 4, wherein the air detection circuit includes a comparator configured to analyze the filtered output to determine whether medical fluid or air is present during the pump stroke.

6. The medical fluid delivery system of claim 5, wherein the air detection circuit includes a counter, and wherein an output from the comparator to the counter goes high and a count at the counter is incremented if medical fluid is determined to be present during the pump stroke.

7. The medical fluid delivery system of claim 6, wherein the output from the comparator to the counter goes low and the count at the counter is not incremented if air is determined to be present during the pump stroke.

8. The medical fluid delivery system of claim 6, wherein the control unit is configured to multiply the count by a known volume for the stroke to determine at least one volume of fluid pumped over multiple strokes of the fluid pump.

9. The medical fluid delivery system of claim 8, wherein the at least one volume of fluid pumped is at least one of a patient drain volume or a patient fill volume for a peritoneal dialysis (“PD”) treatment.

10. The medical fluid delivery system of claim 9, wherein the at least one volume of fluid pumped includes both the patient drain volume and the patient fill volume, and wherein the control unit is configured to subtract the patient fill volume from the patient drain volume to determine an amount of ultrafiltration removed from a patient during the PD treatment.

11. The medical fluid delivery system of claim 5, wherein the control unit includes at least one processor, and wherein the at least one processor is configured to increment a count if an output from the comparator indicates that medical fluid is determined to be present during the pump stroke.

12. The medical fluid delivery system of claim 5, wherein the air detection circuit includes a reset input to the counter, the reset input configured to reset the count to zero prior to at least one of a patient drain or a patient fill for a peritoneal dialysis treatment.

13. The medical fluid delivery system of claim 1, wherein the control unit includes at least one processor and at least one memory configured to use the output from the pressure sensor to determine whether medical fluid or air is present during the pump stroke.

14. The medical fluid delivery system of claim 1, wherein the control unit is configured to analyze peak to peak values of the output from the pressure sensor to determine whether medical fluid or air is present during the pump stroke.

15. The medical fluid delivery system of claim 14, wherein the output from the pressure sensor is a raw output, and wherein the peak to peak values are from the raw output.

16. The medical fluid delivery system of claim 14, wherein of the output from the pressure sensor is a sinusoidal output, and wherein the peak to peak values are from the sinusoidal output.

17. The medical fluid delivery system of claim 14, wherein the control unit is configured to determine that air is present during the pump stroke if a threshold decrease in peak to peak values of the output from the pressure sensor is detected.

18. The medical fluid delivery system of claim 14, wherein the control unit is configured to determine that medical fluid is present during the pump stroke if a threshold decrease in peak to peak values of the output from the pressure sensor is not detected.

19. The medical fluid delivery system of claim 1, wherein the output from the pressure sensor is for an upstream portion of the pump stroke during a patient drain or a patient fill.

20. The medical fluid delivery system of claim 1, wherein the output from the pressure sensor is for a downstream portion of the pump stroke during a patient drain or a patient fill.

21. A medical fluid method comprising:
determining a delta between peak to peak values of an output of a pressure sensor located upstream or downstream of a fluid pump performing a pump stroke;
comparing the determined delta to a threshold delta between peak to peak values; and
determining that medical fluid is present during the pump stroke if the determined delta is greater than the threshold delta.

22. The medical fluid method of claim 21, which includes determining that air or a combination of air and medical fluid is present during the pump stroke if the determined delta is less than the threshold delta.
